(19) United States
(12) Patent Application Publication
(10) Pub. No.: US 2025/0195192 A1
(43) Pub. Date: Jun. 19, 2025

(54) WINGED CAPSULE DEVICES AND METHODS

(71) Applicant: ELANCO US INC., Greenfield, IN (US)

(72) Inventors: Jianbin LI, Greenfield, IN (US); James R. KILLORY, Greenfield, IN (US); Jeremy L. HEMINGWAY, Greenfield, IN (US)

(21) Appl. No.: 18/852,756

(22) PCT Filed: Mar. 29, 2023
(86) PCT No.: PCT/US2023/065096

§ 371 Date: Sep. 30, 2024

Related U.S. Application Data
(60) Provisional application No. 63/325,783, filed on Mar. 31, 2022.

(51) Int. Cl. A61D 7/00

(52) U.S. Cl. CPC A61D 7/00

(57) ABSTRACT

Exemplary winged capsules for delivery of a substance to an animal is provided. The winged capsules can include a capsule defining a central axis and an inner space adapted to contain the substance. A capsule retainer can include a central wing portion connected to the capsule and wings connected to and extending laterally from the central wing portion. At least a first wing of the wings can include: a first lateral edge rib; a second lateral edge rib; and an intermediate longitudinal rib disposed between the first lateral edge rib and the second lateral edge rib. The intermediate longitudinal rib can extend laterally further away from the central axis than the first lateral edge rib and the second lateral edge rib. In one aspect, a thickness of the first wing can continuously change from a first rib end of the first wing to a second rib end of the first wing. In another aspect, a lateral edge rib can extend continuously from the central wing portion to a distal end of the first wing.

### CROSS-REFERENCE TO RELATED APPLICATIONS

This application claims the benefit under 35 USC § 119 (e) of U.S. Provisional Application Ser. No. 63/325,783, filed on Mar. 31, 2022, the entire disclosure of which is incorporated herein by reference.

### FIELD OF THE DISCLOSURE

In some aspects, the disclosure relates generally to winged capsule devices and methods with improvements to better withstand geometric stress during bending.

### BACKGROUND OF THE DISCLOSURE

Controlled release formulations of monensin (as monensin sodium) can be contained in a plastic capsule equipped with retaining wings. Slowly releasing monensin from an opening at the distal end of the capsule, the capsule can remain effective for an average of 95 days after administration in lactating dairy cattle. The capsule can also be used to dispense other formulations, which can have shorter or longer administration periods, for example in the range of 30-180 days.

To administer monensin, the wings of the winged capsule are folded down along the body of the capsule and then placed in an administration tool. The administration tool containing the winged (folded) capsule can be introduced into the animal's mouth, then the administration tool is triggered by pressing a plunger to expel the winged capsule into the pharynx of the animal. The capsule then passes through the esophagus and into the reticulorumen, at which time the wings, which are flexible and resilient, expand to keep the winged capsule in the reticulorumen and prevent regurgitation of the winged capsule by the animal.

The wings can be flexible and resilient to enable insertion and self-expansion, and to maintain the capsule in position while also preventing tissue damage. The capsule can have a rounded top, or dome, and the wings can be attached to the center of the dome. The rounded shape of the dome along a plane traversing the wings, or folding plane, can facilitate folding of the wings, therefore the attachment surface, or joint, maintaining a small profile suitable for insertion into the pharynx. The joint can comprise a small amount of material forming a neck between the wings and the surface of the dome.

### SUMMARY

Winged capsuled devices sized and configured to deliver a substance to an animal are provided herein. Associated methods are also provided herein.

In an Example 1, an exemplary winged capsule for delivery of a substance to an animal is provided. The winged capsule can include a capsule defining a central axis and an inner space adapted to contain the substance. The capsule can include an aperture adapted to deliver the substance to the animal. The capsule can include a capsule retainer having a central wing portion connected to the capsule and wings connected to and extending laterally from the central wing portion. At least a first wing of the wings can include a first lateral edge rib; a second lateral edge rib; and an intermediate longitudinal rib disposed between the first lateral edge rib and the second lateral edge rib. The intermediate longitudinal rib can extend laterally further away from the central axis than the first lateral edge rib and the second lateral edge rib.

In an Example 2, a thickness of the first wing can change from the central wing portion to a first end of the first wing.

In an Example 3, the first wing continuously tapers in a thickness direction.

In an Example 4, the first lateral edge rib terminates at a first rib end, the first wing further including a distal end having a distal edge rib, the distal edge rib terminating at a second rib end, and a thickness of the first wing changes from the first rib end to the second rib end.

In an Example 5, the first wing includes a distal end having a distal width and an intermediate section between the distal end and the central wing portion and the intermediate section can have a width narrowing from a width adjacent the central wing portion to the distal width.

In an Example 6, the intermediate longitudinal rib terminates at a first rib end, and the first rib end is disposed between the intermediate section and the distal end.

In an Example 7, the second lateral edge rib terminates at a second rib end, and the second rib end is disposed between the intermediate section and the distal end.

In an Example 8, the intermediate longitudinal rib terminates at a first rib end, and the first rib end is disposed apart from the central axis by a lateral distance of 60 mm to 80 mm.

In an Example 9, the first lateral edge rib terminates at a first rib end, the second lateral edge rib terminates at a second rib end, and the first rib end and the second rib end are disposed apart from the central axis by a lateral distance of 55 mm to 70 mm.

In an Example 10, the first wing further includes a distal end having a distal edge rib, the distal edge rib having a lateral dimension of 5 mm to 20 mm.

In an Example 11, an exemplary winged capsule for delivery of a substance to an animal is provided. The winged capsule can include a capsule defining a central axis and an inner space adapted to contain the substance. The capsule can include an aperture adapted to deliver the substance to the animal. A capsule retainer can be provided, including a central wing portion connected to the capsule and wings connected to and extending laterally from the central wing portion. At least a first wing of the wings can include a first lateral edge rib extending from the central wing portion and terminating at a first rib end and a distal end having a distal edge rib, the distal edge rib terminating at a second rib end. A thickness of the first wing can continuously change from the first rib end to the second rib end.

In an Example 12, the first wing further includes: a second lateral edge rib extending from the central wing portion and an intermediate longitudinal rib extending from the central wing portion and disposed between the first lateral edge rib and the second lateral edge rib.

In an Example 13, the first rib end can be disposed apart from the central axis by a lateral distance of 55 mm to 70 mm.

In an Example 14, a winged capsule for delivery of a substance to an animal is provided. The winged capsule can include a capsule defining a central axis and an inner space adapted to contain the substance. The capsule can include an aperture adapted to deliver the substance to the animal. A capsule retainer can be provided, including a central wing portion connected to the capsule and wings connected to and extending laterally from the central wing portion to a distal end. At least a first wing of the wings can include a lateral edge rib extending continuously from the central wing portion to the distal end.

In an Example 15, the lateral edge rib has a uniform thickness between the central wing portion and the distal end.

In an Example 16, the lateral edge rib extends continuously from the central wing portion, to the distal end, and back to the central wing portion.

In an Example 17, the distal end has a distal width and the first wing further includes an intermediate section between the distal end and the central wing portion. The intermediate section can have a width narrowing from a width adjacent the central wing portion to the distal width.

In an Example 18, the lateral edge rib extends continuously from the central wing portion, along the intermediate section, to the distal end, back along the intermediate section, and back to the central wing portion.

In an Example 19, the first wing further includes an intermediate longitudinal rib disposed between opposite sides of the lateral edge rib.

In an Example 20, the intermediate longitudinal rib terminates at a first rib end, and the first rib end is disposed apart from the central axis by a lateral distance of 55 mm to 70 mm.

In an Example 21, an exemplary first wing can include a first lateral edge rib; a second lateral edge rib; and an intermediate longitudinal rib disposed between the first lateral edge rib and the second lateral edge rib. The intermediate longitudinal rib can extend laterally further than the first lateral edge rib and the second lateral edge rib. The first wing can include any of the other features of Examples 1-20.

In an Example 22, a method of delivering a substance to an animal using the winged capsule of any of Examples 1-20 is provided.

### DESCRIPTION OF THE DRAWINGS

FIG. 1 is a side view of an illustrative winged capsule in accordance with embodiments of the subject matter disclosed herein;

FIG. 2 is a top perspective view of the winged capsule of FIG. 1;

FIG. 3 is a top view of a first wing of the winged capsule of FIG. 1;

FIG. 4 illustrates bending stresses to which winged capsules, including the winged capsule of FIG. 1, are subjected at various distances from their central axes upon application of a point load;

FIG. 5 is a side view of another illustrative winged capsule in accordance with embodiments of the subject matter disclosed herein;

FIG. 6 is a top perspective view of the winged capsule of FIG. 5;

FIG. 7 is a top view of a first wing of the winged capsule of FIG. 5;

FIG. 8 illustrates bending stresses to which winged capsules, including the winged capsule of FIG. 5, are subjected at various distances from their central axes upon application of a point load;

FIG. 9 illustrates load versus deformation curves for computational models of winged capsules, including the winged capsules of FIGS. 1 and 5;

FIG. 10 is a side view of a computational model of a first wing of a first different winged capsule being subjected to downward point load;

FIG. 11 is a top perspective view of the computational model of FIG. 10;

FIG. 12 is a side view of a computational model of a first wing of a second different winged capsule being subjected to downward point load;

FIG. 13 is a top perspective view of the computational model of FIG. 12;

FIG. 14 is a side view of a computational model of a first wing of the winged capsule of FIG. 1 being subjected to a downward point load;

FIG. 15 is a top perspective view of the computational model of FIG. 14;

FIG. 16 is a side view of a computational model of a first wing of the winged capsule of FIG. 5 being subjected to a downward point load; and

FIG. 17 is a top perspective view of the computational model of FIG. 16.

In the drawings, corresponding reference characters indicate corresponding parts, functions, and features throughout the several views. Although the drawings represent embodiments of various features and components according to the present invention, the drawings are not necessarily to scale and certain features may be exaggerated in order to better illustrate and explain the present invention. However, the invention is not limited to the precise arrangements and instrumentalities of the embodiments shown in the drawings.

### DETAILED DESCRIPTION OF DISCLOSED EMBODIMENTS

For the purposes of promoting an understanding of the principles of the invention, reference will now be made to the embodiments illustrated in the drawings, which are described below. The embodiments disclosed below are not intended to be exhaustive or limit the invention to the precise form disclosed in the following detailed description. Rather, the embodiments are chosen and described so that others skilled in the art may utilize their teachings. It will be understood that no limitation of the scope of the invention is thereby intended. The invention includes any alterations and further modifications in the illustrated devices and described methods and further applications of the principles of the invention as set forth in the claims.

Some parts described below with reference to the figures are common to multiple embodiments and variations thereof. Throughout the figures the same reference numerals are given to the common parts and the descriptions of the common parts are not repeated for simplicity. Furthermore, unless specifically stated, the depictions of the embodiments and variations thereof are made to scale although the scale can vary between embodiments.

After manufacture, multiple winged capsules containing a substance can be placed inside packaging, typically a bag. It has been found that in the process of packaging the winged capsule and transporting the bags, wings might bend near the joint past the point of material yield, at which point they no longer recover their functional shape. The bent wings might also break and separate from the bolus. Further, the winged capsules can be subjected to cold conditions during shipping and handling (for example, −20° C. to 0° C.) and as a result can suffer end fractures, particularly after drop impact. The damaged devices must be discarded taking care that they are properly disposed of in light of their content. An economical solution is needed to prevent damage to winged capsules to reduce waste and disposal costs.

Winged capsules in accordance with aspects described herein advantageously include one or more wings that are subjected to relatively uniform bending stresses along their length, and avoid high peak bending stresses and abrupt stress changes. In some aspects, these advantages are provided by rib arrangements and/or tapering thicknesses of such wings. In some aspects, the wings are less prone to breakage during packaging, transport, and while positioned in an animal.

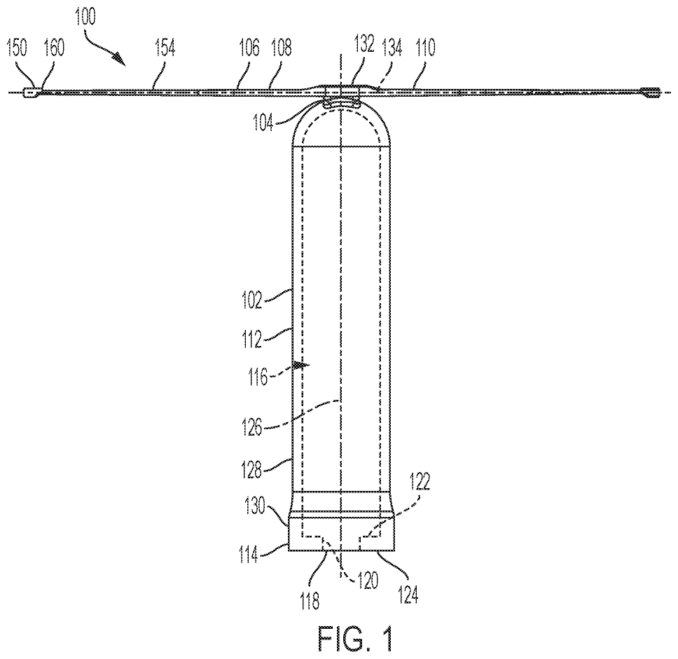

FIGS. 1-3 depict an illustrative winged capsule 100 for delivery of a substance to an animal, in accordance with aspects described herein. Referring specifically to FIG. 1, winged capsule 100 includes a capsule 102, a neck 104 (shown in FIG. 1) extending from capsule 102, and a capsule retainer 106. The capsule retainer 106 includes a first wing 108 and a second wing 110 that flex, or are foldable, and are positioned near the capsule 102 to facilitate placement in packaging, such as a bag, and delivery of the winged capsule 100 to an animal.

Capsule 102 has a tubular wall 112 and a bottom wall 114, the tubular wall 112 and the bottom wall 114 define an inner space 116, and the bottom wall 114 has an aperture 118 adapted to deliver the substance to the animal. A rim portion 120 of the bottom wall 114 surrounds aperture 118. Bottom wall 114 has an inner surface 122 and an outer surface 124. A substance (not shown) is placed in capsule 102 and configured for discharge over extended periods of time through aperture 118. The substance can comprise monensin or other medicaments, vitamins, and any other substance suitable for delivery over an extended period of time. The extended period of time could be equal to or more than 30, 60, 90, 95, 120, 140, 150 or 154 days.

Capsule 102 can have a cylindrical shape. Stated another way, capsule 102 includes a longitudinal axis 126, which defines a central axis of winged capsule 100. Capsule 102 can have a circular cross-sectional shape perpendicular to longitudinal axis 126. Alternatively, capsule 102 can have a different shape, or a different cross-sectional shape perpendicular to longitudinal axis 126, such as an oval or the like.

Capsule 102 can include a top portion 128 and a bottom portion 130. During manufacture of winged capsule 100, the substance can be inserted into top portion 128 of capsule 102 and then bottom portion 130 is affixed to top portion 128 to retain the substance therein. Alternatively, the substance can be inserted through a lateral opening (not shown) on capsule 102. Top portion 128 of capsule 102 can be affixed to or monolithically constructed with neck 104, and neck 104 can be affixed to or monolithically constructed with capsule retainer 106.

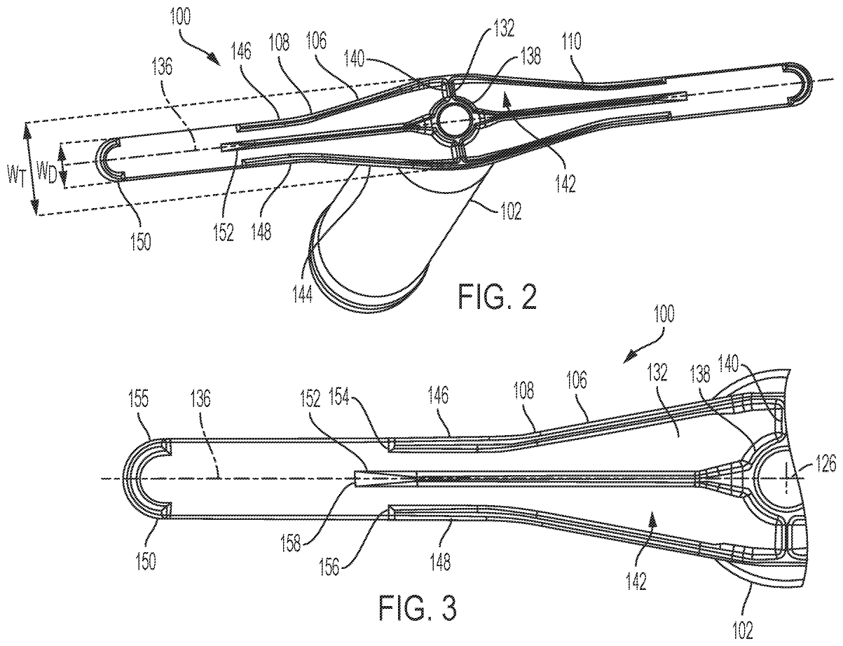

Referring more generally to FIGS. 1-3, capsule retainer 106 includes a central wing portion 132 connected to capsule 102 and first wing 108 and second wing 110 connected to central wing portion 132. First wing 108 and second wing 110 are connected to and extend laterally from central wing portion 132 along a wing plane 134 (shown in FIG. 1). Stated another way, a longitudinal axis 136 (shown in FIGS. 2 and 3) of first wing 108 and second wing 110 extends laterally from central wing portion 132. Central wing portion 132 includes a central rib 138 and a transverse rib 140 (both shown in FIG. 2), and first wing 108 and second wing 110 are disposed on opposite sides of transverse rib 140. First wing 108 and second wing 110 include a top surface 142 that defines wing plane 134 and a plurality of ribs extending therefrom, as described in further detail below.

Referring specifically to FIG. 2, central wing portion 132 has a maximum central width W_{T} orthogonal to longitudinal axis 136 and wings 108, 110 have a distal width W_{D}. An intermediate section 144 tapers from W_{T} to W_{D}. In some aspects, W_{D} is in a range of 35% to 65% of W_{T}. W_{D} can be, more specifically, in a range of 40% to 60% of W_{T}, and even more specifically in a range of 45% to 55% of W_{T}. If capsule 102 has a cylindrical shape, the width of capsule 102 is its diameter, and W_{T} can be at least 55% of the diameter. W_{T} can be, more specifically, at least 60%, 80%, and even more specifically at least 85% of the diameter.

As described briefly above, first wing 108 and second wing 110 include a plurality of ribs. Referring specifically to FIG. 3, the exemplary ribs of first wing 108 will be described in further detail. Although the ribs of second wing 110 are not described in further detail, it is understood that the ribs of second wing 110 can be the same as or similar to the ribs of first wing 108. The plurality of ribs of first wing 108 include a first lateral edge rib 146, a second lateral edge rib 148, a distal edge rib 150, and an intermediate longitudinal rib 152 between first lateral edge rib 146 and second lateral edge rib 148.

With continued reference to FIG. 3, one or more of the plurality of ribs of first wing 108 can include dimensions that facilitate subjecting the first wing 108 to relatively uniform bending stresses along its length, and to avoid high peak bending stresses and abrupt stress changes. For example, and as illustrated, first lateral edge rib 146 can terminate at a first rib end 154 disposed between intermediate section 144 and a distal end 155 of first wing 108. First rib end 154 can be disposed apart from central axis 126 by a lateral distance (that is, a distance parallel to longitudinal axis 136) of 55 mm to 70 mm, more specifically 58 mm to 67 mm, or even more specifically 60 mm to 65 mm. As another example and as illustrated, second lateral edge rib 148 can terminate at a second rib end 156 disposed between intermediate section 144 and distal end 155. Second rib end 156 can be disposed apart from central axis 126 by a lateral distance of 55 mm to 70 mm, more specifically 58 mm to 67 mm, or even more specifically 60 mm to 65 mm. As another example, and as illustrated, intermediate longitudinal rib 152 can extend laterally further away from central axis 126 than first lateral edge rib 146 and/or second lateral edge rib 148. More specifically, intermediate longitudinal rib 152 can terminate at a third rib end 158 disposed between intermediate section 144 and distal end 155. Third rib end 158 can be disposed apart from central axis 126 by a lateral distance of 60 mm to 80 mm, more specifically 63 mm to 71 mm, or even more specifically 65 mm to 69 mm. As yet another example, distal edge rib 150 can have a lateral dimension of 5 mm to 20 mm, or more specifically 8 mm to 15 mm. As a further example and with additional reference to FIG. 1, first wing 108 can continuously taper in a thickness direction (that is, a direction parallel to central axis 126). Stated another way, the thickness of first wing 108 can change from central wing portion 132 to a first end of first wing 108. More specifically, the thickness of first wing 108 can change from first rib end 154 to a fourth rib end 160 of distal edge rib 150. Even more specifically, first wing 108 can continuously taper from a thickness of about 2.3 mm near the first rib end 154 to a thickness of about 1.0 mm near the fourth rib end 160.

As most clearly shown in FIG. 2, lateral edge ribs 146 and 148 have a greater height near transverse rib 140, and the height decreasingly tapers extending laterally away from the transverse rib 140. Without being bound by theory, it is believed that the height difference provides additional rigidity/support to central wing portion 132, particularly over neck 104. A similar principle is applied to intermediate longitudinal rib 152 near central rib 138. More specifically, intermediate longitudinal rib 152 has a greater width near central rib 138, and the width decreasingly tapers extending laterally away from central rib 138.

Winged capsule 100 can be injection molded from materials including, for example, polypropylene, polyethylene, more specifically high-density polyethylene. Exemplary polypropylene materials include those with the characteristics listed below, although other injection molding grade polymers can also be used, such as those listed hereinabove. In one aspect, these materials are more resilient to flexure than materials used in prior devices and thus help reduce damage as described herein.

| Property | Range | Units | Test Method |
|---|---|---|---|
| Density (23° C.) | .880-.920 | g/cc | ISO 1183 |
| Tensile modulus | 1000-1600 | MPa | ISO 527 |
| Tensile stress at yield | 20-30 | MPa | ISO 527 |
| Elongation at yield | 3.5-7.5 | % | ISO 527 |
| Shore D Hardness | 60-75 | | ISO 868/7619 |

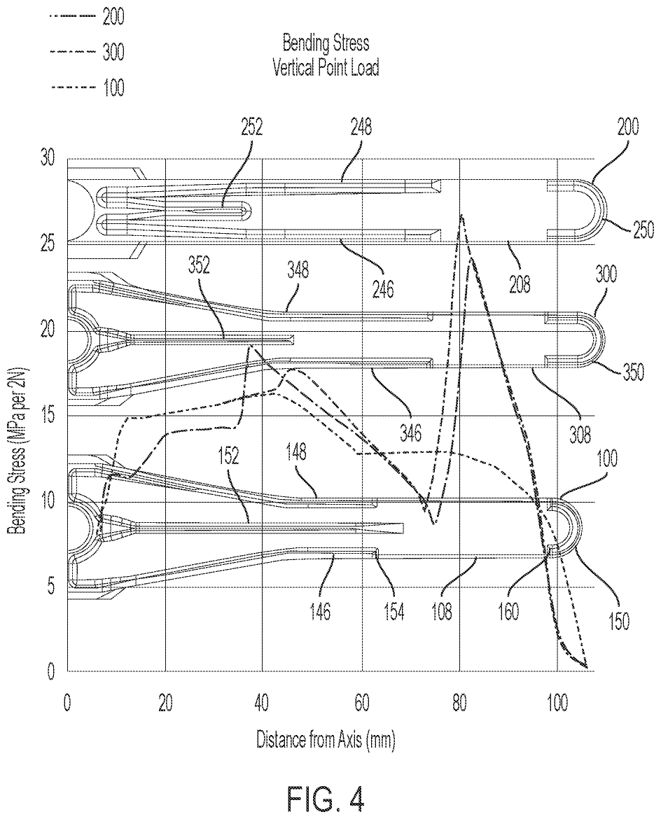

FIG. 4 illustrates bending stresses curves, more specifically bending stresses to which winged capsules are subjected. More specifically, FIG. 4 illustrates bending stresses to which first wing 108 of winged capsule 100 is subjected at various distances from central axis 126 (shown elsewhere). FIG. 4 also illustrates bending stresses to which a first wing 208 of a first different winged capsule 200 and a first wing 308 of a second different winged capsule 300 are subjected at various distances from their central axes. First wings 108, 208, and 308 of winged capsules 100, 200, and 300, respectively, are also superimposed on the curves.

The dimensions of the winged capsules 100, 200, and 300 shown in FIG. 4 can be determined based on scale provided by the x-axis. Additionally, first wing 108 of winged capsule 100 can have a thickness that continuously tapers from 2.3 mm at first rib end 154 to 1.0 mm at fourth rib end 160, and ribs 146, 148, 150, and 152 have a general height of about 0.35 mm. First wing 208 of winged capsule 200 has a thickness that briefly tapers near lateral edge ribs 246 and 248 from 1.9 mm to 1.0 mm, and ribs 246, 248, 250, and 252 have a general height of about 0.7 mm. First wing 308 of winged capsule 300 has a uniform thickness of about 1.0 mm, and ribs 346, 348, 350, and 352 have a general height of about 0.35 mm. Alternatively, the thickness can taper in a discontinuous manner as described above.

The bending stress curves of FIG. 4 illustrate calculated bending stresses upon application of a downward point load to the distal end of each winged capsule 100, 200, and 300. The bending stresses were calculated assuming a linear elastic material response and small deflections, and the results illustrate trends and a provide a comparison between responses of the winged capsules 100, 200, and 300 to a point load. Advantageously and surprisingly, FIG. 4 illustrates that winged capsule 100 is subjected to relatively uniform bending stresses, relatively low peak bending stresses, and few abrupt stress changes compared to winged capsules 200 and 300, particularly between lateral edge ribs 146 and 148 and distal edge rib 150.

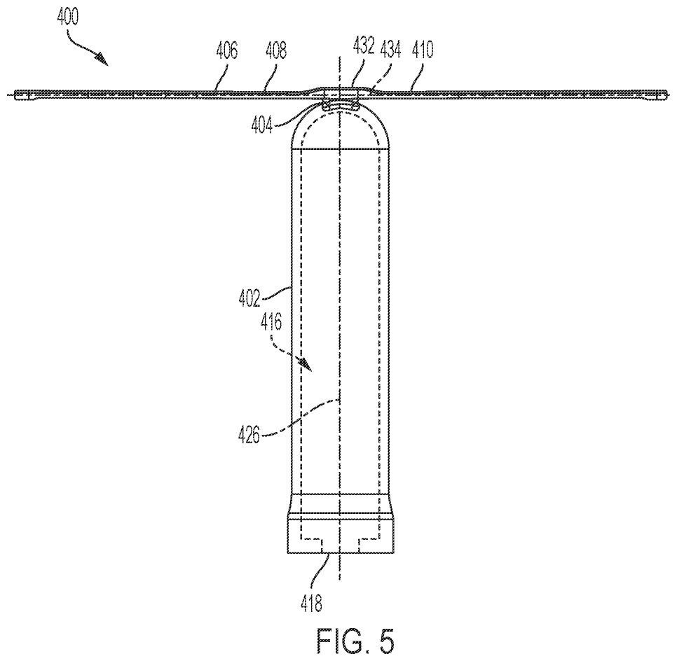

FIGS. 5-7 depict another illustrative winged capsule 400 for delivery of a substance to an animal, in accordance with embodiments of the subject matter disclosed herein. Winged capsule 400 includes some of the features of winged capsule 100 and, in one aspect, can be constructed of the same materials as winged capsule 100. More specifically, winged capsule 400 includes a capsule 402, a neck 404 (shown in FIG. 5) extending from capsule 402, and a capsule retainer 406. Capsule retainer 406 includes a first wing 408 and a second wing 410 that flex, or are foldable, and are positioned near the capsule 402 to facilitate placement in packaging, such as a bag, and delivering winged capsule 400 to an animal. Additionally, capsule 402 includes an inner space 416 and an aperture 418 (both shown in FIG. 5) adapted to deliver the substance to the animal. A substance can be placed in capsule 402 and configured for discharge over extended periods of time through aperture 418. Capsule 402 includes a longitudinal axis 426 (shown in FIG. 5), which defines a central axis of winged capsule 400.

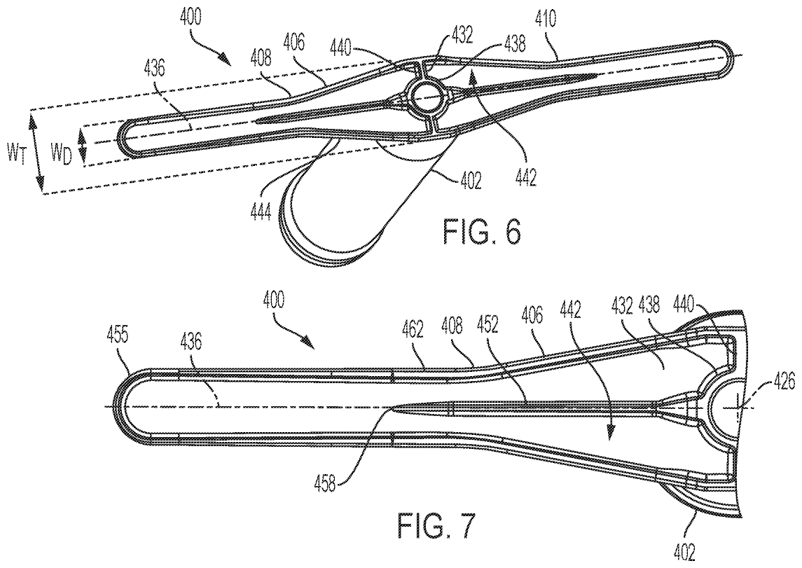

Capsule retainer 406 shares certain features of capsule retainer 106 of winged capsule 100. That is, capsule retainer 406 includes a central wing portion 432 connected to capsule 102 and first wing 408 and second wing 410 connected to central wing portion 432. First wing 408 and second wing 410 are connected to and extend laterally from central wing portion 432 along a wing plane 434 (shown in FIG. 5). Stated another way, a longitudinal axis 436 (shown in FIGS. 6 and 7) of first wing 408 and second wing 410 extends laterally from central wing portion 432. Central wing portion 432 includes a central rib 438 and a transverse rib 440 (both shown in FIG. 6), and first wing 408 and second wing 410 are disposed on opposite sides of transverse rib 440. First wing 408 and second wing 410 include a top surface 442 that defines wing plane 434 and a plurality of ribs extending therefrom, as described in further detail below.

Referring specifically to FIG. 6, central wing portion 432 has a maximum central width W_{T} orthogonal to longitudinal axis 436 and wings 408 and 410 have a distal width W_{D}. An intermediate section 444 tapers from W_{T} to W_{D}. In some embodiments, W_{D} is in a range of 35% to 65% of W_{T}. W_{D} can be, more specifically, in a range of 40% to 60% of W_{T}, and even more specifically in a range of 45% to 55% of W_{T}. If capsule 402 has a cylindrical shape, the width of capsule 402 is its diameter, and W_{T} can be at least 55% of the diameter. W_{T} can be, more specifically, at least 60%, 80%, and even more specifically at least 85% of the diameter.

As described above, first wing 408 and second wing 410 include a plurality of ribs. Referring specifically to FIG. 7, the ribs of first wing 408 will be described in further detail. Although the ribs of second wing 410 are not described in further detail, it is understood that the ribs of second wing 410 can be the same as or similar to the ribs of first wing 408. The plurality of ribs of first wing 408 include an intermediate longitudinal rib 452 and, in contrast to first wing 108 of winged capsule 100, a single, continuously extending lateral edge rib 462. Illustratively, the lateral edge rib 462 extends continuously (that is, without interruption) from central wing portion 432, along intermediate section 444, to distal end 455 of first wing 408, back along intermediate section 444, and back to central wing portion 432. It is understood that lateral edge rib 462 can, alternatively, extend discontinuously from central wing portion 432. Illustratively, intermediate longitudinal rib 452 is disposed between opposite sides of lateral edge rib 462.

With continued reference to FIG. 7, one or more of the plurality of ribs of first wing 408 can include dimensions that facilitate subjecting the first wing 408 to relatively uniform bending stresses along its length, and to avoid high peak bending stresses and abrupt stress changes. For example, and as illustrated, lateral edge rib 462 has a uniform thickness between central wing portion 432 and distal end 455. The uniform thickness can be, for example, from 1.7 mm to 1.9 mm. As another example, and as illustrated, intermediate longitudinal rib 452 can terminate at a first rib end 458, and first rib end 458 can be disposed apart from central axis 426 by a lateral distance of 55 mm to 70 mm, more specifically 58 mm to 67 mm, or even more specifically 60 mm to 65 mm.

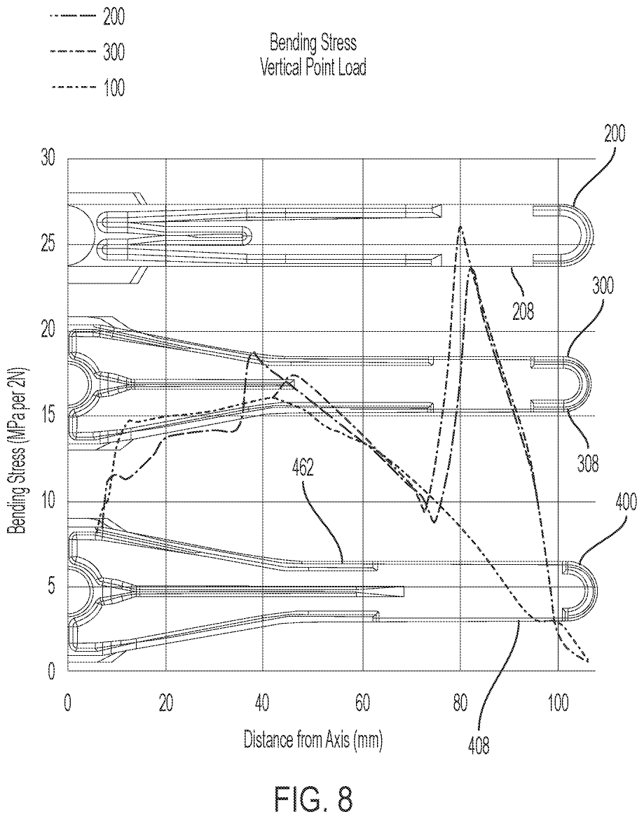

FIG. 8 illustrates bending stresses curves, more specifically bending stresses to which winged capsules are subjected. FIG. 8 illustrates bending stresses to which first wing 408 of winged capsule 400 is subjected at various distances from central axis 426 (shown elsewhere). FIG. 8 also illustrates bending stresses to which first wing 208 of winged capsule 200 and first wing 308 of winged capsule 300 are subjected at various distances from their central axes. First wings 408, 208, and 308 of winged capsules 400, 200, and 300, respectively, are also superimposed on the curves.

In one aspect, the dimensions of winged capsule 400 can be determined based on the scale provided by the x-axis. Additionally, first wing 408 of winged capsule 400 is shown to have a uniform thickness of 2.3 mm apart from lateral edge rib 462, and lateral edge rib 462 has a uniform thickness of 2.4 mm. Some of the dimensions of winged capsules 200 and 300 can be determined, for example, based on scale provided by the x-axis, and other exemplary dimensions of winged capsules 200 and 300 are provided above.

The curves of FIG. 8 illustrate bending stresses upon application of a downward point load to the distal end of each winged capsule 400, 200, and 300. The exemplary bending stresses were calculated assuming a linear elastic material response and small deflections. Advantageously and surprisingly, FIG. 8 illustrates that winged capsule 400 is subjected to relatively uniform bending stresses, relatively low peak bending stresses, and few abrupt stress changes compared to winged capsules 200 and 300.

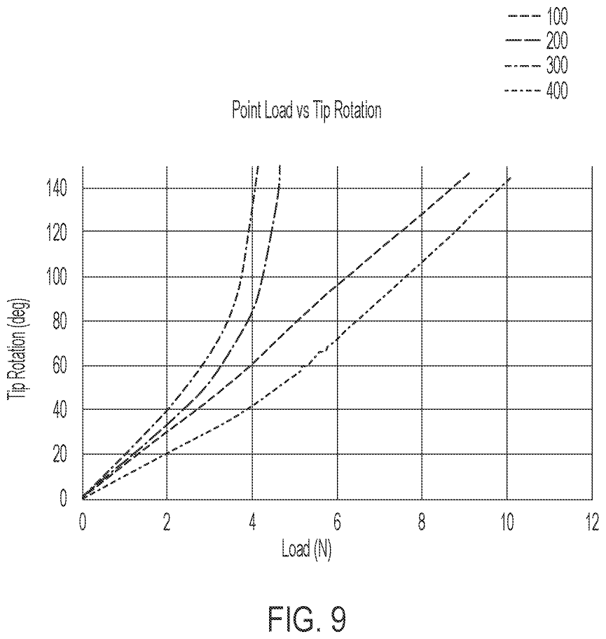

Computational models were developed, using Abaqus Finite Element Analysis (FEA) software available from ABAQUS Inc., for winged capsules 100, 200, 300, and 400 to simulate load conditions. This software and other FEA software has been recognized as an important tool for evaluating medical devices. See, for example, Liu, Cheryl; FDA Recognizes Simulation Essential to Evaluate Medical Devices; Advanced Materials & Process; April 2013, pp. 32-33. See also, for example, U.S. Food & Drug Administration; Credibility of Computational Models Program: Research on Computational Models and Simulation Associated with Medical Devices; www.fda.gov; and Abaqus Version 6.6 Documentation; classes.engineering.wustl.edu. In contrast to the bending stress curves of FIGS. 4 and 8, utilized elastic-plastic material models and non-linear finite element techniques. Half symmetry, three-dimensional models were utilized, and the central wing portion of each winged capsule was held fixed to isolate the load to the first wing. Resulting load versus deformation curves, more specifically load versus wing distal end rotation curves are shown in FIG. 9. As illustrated, winged capsules 100 and 400 are subjected to relatively low distal end rotation compared to winged capsules 200 and 300.

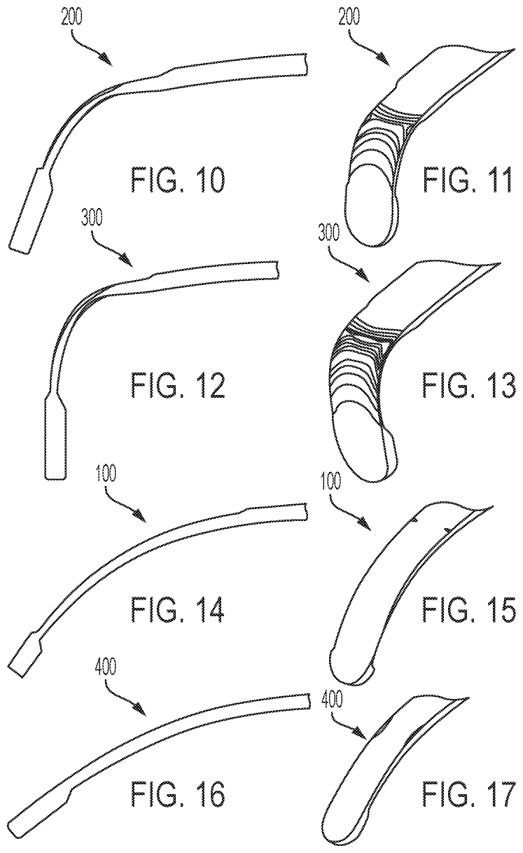

The computational models are illustrated at a downward point load of 3.6N in FIGS. 10-17. FIGS. 10 and 11 illustrate winged capsule 200, FIGS. 12 and 13 illustrate winged capsule 300, FIGS. 14 and 15 illustrate winged capsule 100, and FIGS. 16 and 17 and 15 illustrate winged capsule 400. As illustrated, winged capsules 200 and 300 are subjected to relatively high peak strains in regions 270 and 370, respectively, adjacent their distal ends. Winged capsules 100 and 400, in contrast are subjected to relatively low peak strains. The computational models confirm the trends suggested by the bending stress curves of FIGS. 4 and 8. The results also indicate that winged capsules 100 and 400 are less likely to yield than winged capsules 200 and 300 at similar loads.

Except where a contrary intent is expressly stated, the terms “comprises,” “comprising,” “containing,” and “having” and the like mean “includes,” “including,” and the like, and are generally interpreted to be open ended transition terms. The recitation of components, structures, steps, or the like specifically listed following an open-ended transition term in no way limit such claim to the components, structures, steps, or the like specifically listed. The terms “consisting of” or “consists of” are closed transition terms.

Except where a contrary intent is expressly stated, the terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps can be performed, and certain of the stated steps can possibly be omitted and/or certain other steps not described herein can possibly be added to the method.

Except where a contrary intent is expressly stated, terms are used in their singular form for clarity and are intended to include their plural form.

Occurrences of the phrase “in one embodiment,” or “in one aspect,” herein do not necessarily all refer to the same embodiment or aspect.

While this invention has been described as having designs illustrated by embodiments and examples, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

1. A winged capsule for delivery of a substance to an animal, the winged capsule comprising:
a capsule defining a central axis and an inner space adapted to contain the substance, and the capsule comprising an aperture adapted to deliver the substance to the animal;
a capsule retainer comprising a central wing portion connected to the capsule and wings connected to and extending laterally from the central wing portion, at least a first wing of the wings comprising:
a first lateral edge rib;
a second lateral edge rib; and
an intermediate longitudinal rib disposed between the first lateral edge rib and the second lateral edge rib, the intermediate longitudinal rib extending laterally further away from the central axis than the first lateral edge rib and the second lateral edge rib.

2. The winged capsule of claim 0, wherein a thickness of the first wing changes from the central wing portion to a first end of the first wing.

3. The winged capsule of claim 0, wherein the first wing continuously tapers in a thickness direction.

4. The winged capsule of claim 0, wherein the first lateral edge rib terminates at a first rib end, the first wing further comprising a distal end having a distal edge rib, the distal edge rib terminating at a second rib end, and a thickness of the first wing changes from the first rib end to the second rib end.

5. The winged capsule of claim 0, wherein the first wing comprises a distal end having a distal width and an intermediate section between the distal end and the central wing portion, the intermediate section having a width narrowing from a width adjacent the central wing portion to the distal width.

6. The winged capsule of claim 0, wherein the intermediate longitudinal rib terminates at a first rib end, and the first rib end is disposed between the intermediate section and the distal end.

7. The winged capsule of claim 0, wherein the second lateral edge rib terminates at a second rib end, and the second rib end is disposed between the intermediate section and the distal end.

8. The winged capsule of claim 0, wherein the intermediate longitudinal rib terminates at a first rib end, and the first rib end is disposed apart from the central axis by a lateral distance of 60 mm to 80 mm.

9. The winged capsule of claim 0, wherein the first lateral edge rib terminates at a first rib end, the second lateral edge rib terminates at a second rib end, and the first rib end and the second rib end are disposed apart from the central axis by a lateral distance of 55 mm to 70 mm.

10. The winged capsule of claim 0, wherein the first wing further comprises a distal end having a distal edge rib, the distal edge rib having a lateral dimension of 5 mm to 20 mm.

11. A winged capsule for delivery of a substance to an animal, the winged capsule comprising:
a capsule defining a central axis and an inner space adapted to contain the substance, and the capsule comprising an aperture adapted to deliver the substance to the animal;
a capsule retainer comprising a central wing portion connected to the capsule and wings connected to and extending laterally from the central wing portion, at least a first wing of the wings comprising:
a first lateral edge rib extending from the central wing portion and terminating at a first rib end; and
a distal end having a distal edge rib, the distal edge rib terminating at a second rib end;
wherein a thickness of the first wing continuously changes from the first rib end to the second rib end.

12. The winged capsule of claim 0, wherein the first wing further comprises:
a second lateral edge rib extending from the central wing portion; and
an intermediate longitudinal rib extending from the central wing portion and disposed between the first lateral edge rib and the second lateral edge rib.

13. The winged capsule of claim 0, wherein the first rib end is disposed apart from the central axis by a lateral distance of 55 mm to 70 mm.

14. A winged capsule for delivery of a substance to an animal, the winged capsule comprising:
a capsule defining a central axis and an inner space adapted to contain the substance, and the capsule comprising an aperture adapted to deliver the substance to the animal; and
a capsule retainer comprising a central wing portion connected to the capsule and wings connected to and extending laterally from the central wing portion to a distal end, at least a first wing of the wings comprising a lateral edge rib extending continuously from the central wing portion to the distal end.

15. The winged capsule of claim 0, wherein the lateral edge rib has a uniform thickness between the central wing portion and the distal end.

16. The winged capsule of claim 0, wherein the lateral edge rib extends continuously from the central wing portion, to the distal end, and back to the central wing portion.

17. The winged capsule of claim 0, wherein the distal end has a distal width and the first wing further comprises an intermediate section between the distal end and the central wing portion, the intermediate section having a width narrowing from a width adjacent the central wing portion to the distal width.

18. The winged capsule of claim 0, wherein the lateral edge rib extends continuously from the central wing portion, along the intermediate section, to the distal end, back along the intermediate section, and back to the central wing portion.

19. The winged capsule of claim 0, wherein the first wing further comprises an intermediate longitudinal rib disposed between opposite sides of the lateral edge rib.

20. The winged capsule of claim 0, wherein the intermediate longitudinal rib terminates at a first rib end, and the first rib end is disposed apart from the central axis by a lateral distance of 55 mm to 70 mm.
